(19) United States
(12) Patent Application Publication
(10) Pub. No.: US 2022/0046960 A1
(43) Pub. Date: Feb. 17, 2022

(54) BEVERAGE COMPOSITION AND PROCESS

(71) Applicant: Kurt H. RUPPMAN, Allen, TX (US)

(72) Inventor: Kurt H. RUPPMAN, Allen, TX (US)

(21) Appl. No.: 17/398,827

(22) Filed: Aug. 10, 2021

Related U.S. Application Data
(60) Provisional application No. 63/064,391, filed on Aug. 11, 2020.

(51) Int. Cl. A23L 2/54; A23L 33/175; A23L 2/60; A23L 2/68; A23L 33/16; A23L 33/125; A23L 33/105; A23L 33/15; A23L 33/00; C02F 1/68; B01F 3/04; A61K 33/00; A61K 31/198; A61K 9/00

(52) U.S. Cl. CPC A23L 2/54; B01F 2215/0022; A23L 2/60; A23L 2/68; A23L 33/16; A23L 33/125; A23L 33/105; A23L 33/15; A23L 33/40; C02F 1/68; B01F 3/04439; B01F 3/0446; A61K 33/00; A61K 31/198; A61K 9/0053; A61K 9/0095; B01F 2003/04914; A23L 33/175

(57) ABSTRACT

A beverage comprising a hydrogen-infused, degassed water and a nonessential amino acid are disclosed. The beverage can further include a natural sweetener, a dicarboxylic acid, a tricarboxylic acid, an α-amino acid, an electrolyte, a caffeine composition, and a Vitamin B composition. Also disclosed is a method for making such beverage. The beverage provides benefits to health.

### CROSS-REFERENCE TO RELATED APPLICATIONS

This application claims priority of U.S. Provisional Patent Application Ser. No. 63/064,391, entitled “BOOSTING NITRIC OXIDE PRODUCTION WITH THE ADDITION OF HYDROGEN,” filed on Aug. 11, 2020, the contents of which are incorporated by reference herein in their entirety.

### FIELD OF THE INVENTION

This invention relates to a hydrogen-infused beverages intended to provide multiple health benefits. The invention further relates to methods for manufacturing such beverages.

### BACKGROUND OF THE INVENTION

Nitric oxide is a gas composed of one molecule of nitrogen bonded to one molecule of oxygen. It serves as a critical signaling molecule in the cardiovascular and circulatory system in a human body. This essential molecule signals blood vessels to relax and expand, creating greater efficiency in the body, greater nutrient delivery, and greater oxygenation to every system, organ and tissue in the body. The nitric oxide also helps in improving athletic performance.

Generally, diet and exercise play a large role in stimulating the natural production of nitric oxide. The nitric oxide stimulating foods or foods that are high in dietary nitrites/nitrates with regular exercise helps to promote nitric oxide production in the human body. Unfortunately, in today's world of refined foods these options may not be enough, especially for the athletes and those suffering from certain medical conditions.

Hydrogen water is said to increase energy, reduce inflammation, and reduce recovery times after workouts. Hydrogen water is regular water into which hydrogen has been infused. According to some resources, adding hydrogen gas to the water increases its anti-inflammatory and antioxidant properties when consumed. Hydrogen water has also been touted for its ability to increase energy, slow down the aging process, and improve muscle recovery after a workout.

Although nitric oxide can directly kill pathogens, it is also critical for immune function and can inhibit viral replication. However, nitric oxide in certain quantities or inhaled in an uncontrolled manner as a gas can also induce lethal cellular injury under stressed conditions. In contrast, molecular hydrogen helps regulate nitric oxide production and attenuates many of the harmful effects. In addition, hydrogen water typically referred to in the literature has limited beneficial effects in that are short-lived, lasting less than a few hours.

Therefore, there is a need for a hydrogen water that promotes the controlled production of nitric oxide in the human body for an extended period of time.

### SUMMARY OF THE INVENTION

The present invention relates to a beverage and process for making a beverage for enhancing athletic performance and acquiring other health benefits of nitric oxide for longer duration. In addition, the beverage of the present invention is useful in the treatment of medical conditions that benefit from increased blood flow for longer periods of time.

In some embodiments, the beverage composition comprises molecular hydrogen, a potable fluid and a nonessential amino acid. The potable fluid is preferably water or degassed water, the nonessential amino acid is L-citrulline and/or L-arginine, and level of molecular hydrogen in the beverage composition is in the range of from greater than 1.7 ppm to about 6 ppm by weight, preferably greater than 1.8 ppm to about 6 ppm by weight, and even more preferably from about 2 ppm to 6 ppm by weight. In a most preferred embodiment, the hydrogen is infused into a mixture of the potable fluid and the nonessential amino acid(s).

In some embodiments, the composition comprises the reaction product or product of a mixture of a molecular hydrogen-infused potable fluid, preferably where the potable fluid is water, and more preferably where the potable fluid is degassed water, and a nonessential amino acid. In some embodiments, the potable fluid is water, preferably degassed water, infused with molecular hydrogen in an amount in the range of from 0.5 ppm to 6 ppm by weight. In some embodiments, the nonessential amino acid is L-citrulline and/or L-arginine in the range of from about 0.15 weight percent (wt %) to about 3.0 wt %, based on the total weight of the mixture. In some embodiments, the molecular hydrogen in the composition is in the range of from greater than 1.7 ppm to about 6 ppm by weight, preferably greater than 1.8 ppm to about 6 ppm by weight, and even more preferably from about 2 ppm to 6 ppm by weight

In some embodiments, the composition further comprises one or more of a natural sweetener, a dicarboxylic acid, a tricarboxylic acid, an α-amino acid, an electrolyte, a caffeine composition, and a Vitamin-B composition.

In some embodiments, the natural or artificial sweetener is selected from one or more of stevia, erythritol, monk fruit, acesulfame potassium, allulose, xylitol, and yacon, and natural sweeteners are preferred. In some embodiments, the natural sweetener is added to the mixture in an amount less than or equal to 4 wt %, based on the total weight of the mixture.

In some embodiments, the dicarboxylic acid is selected from one or more of adipic acid, fumaric acid, and malic acid. In some embodiments, the dicarboxylic acid is added to the mixture in an amount less than or equal to 2 wt %, based on the total weight of the mixture.

In some embodiments, the tricarboxylic acid is citric acid. In some embodiments, the tricarboxylic acid is added to the mixture in an amount less than or equal to 2 wt %, based on the total weight of the mixture.

In some embodiments, the α-amino acid is L-glutamine. In some embodiments, the α-amino acid is added to the mixture in an amount less than or equal to 2 wt %, based on the total weight of the mixture.

In some embodiments, the electrolyte is monopotassium phosphate. In some embodiments, the electrolyte is added to the mixture in an amount less than or equal to 2 wt %, based on the total weight of the mixture.

In some embodiments, the caffeine composition is natural green tea. In some embodiments, the caffeine composition is added to the mixture in an amount less than or equal to 2 wt %, based on the total weight of the mixture.

In some embodiments, Vitamin-B composition is selected from one of more of riboflavin (B2), niacin (B3), calcium pantothenate (B5), pyridoxine hydrochloride (B6), biotin (B7), folic acid (B9), and cyanocobalamin (B12). In some embodiments, the Vitamin-B composition is added to the mixture in an amount less than or equal to 2 wt %, based on the total weight of the mixture.

In some embodiments, the process comprises treating water to remove volatiles and/or solids to produce a treated water or degassed water, infusing the treated or degassed water with molecular hydrogen to produce a hydrogen-infused, treated or degassed water, and mixing hydrogen-infused, treated or degassed water, with L-citrulline and/or L-arginine to produce an enhanced water composition or beverage of the invention.

In some embodiments, the potable fluid, preferably where the potable fluid is water, more preferably where the potable fluid is degassed water, is combined simultaneously or co-fed with, molecular hydrogen, through a spray nozzle with orifices small enough for creating micro- or nano-droplets of the composition. In this embodiment, it is preferred that L-citrulline and/or L-arginine are further combined or cofed with the potable fluid and the molecular hydrogen. In another embodiment, the potable fluid and nonessential amino acid(s) are combined to form a mixture that is then combined, co-fed or mixed with molecular hydrogen to form the beverage composition of the invention.

In some embodiments, the process further comprises mixing the enhanced water composition or beverage with one or more of a natural sweetener, a dicarboxylic acid, a tricarboxylic acid, an α-amino acid, an electrolyte, a caffeine composition, and a Vitamin B composition.

The foregoing has outlined rather broadly the features and technical advantages of the present invention in order that the detailed description of the invention that follows may be better understood. Additional features and advantages of the invention will be described hereinafter, which form the subject matter of the claims of the invention. It should be appreciated by those skilled in the art that the conception and specific embodiments disclosed may be readily utilized as a basis for modifying or designing other compositions and/or processes for carrying out the purposes of the present invention. It should also be realized by those skilled in the art that such equivalent constructions do not depart from the spirit and scope of the invention as set forth in the appended claims. The novel features which are believed to be characteristic of the invention, both as to its composition and method of manufacture, together with further objects and advantages will be better understood from the following description.

### BRIEF DESCRIPTION OF THE DRAWINGS

The present disclosure is illustrated by way of example, and not by way of limitation, in the accompanying figure, in which:

FIG. 1 shows a graphical comparison of maximal aerobic power and functional threshold power before and after consuming the inventive beverage disclosed herein;

FIG. 2 shows a graphical comparison of maximal oxygen uptake before and after consuming beverage disclosed herein;

FIG. 3 shows a graphical comparison of average leg extension torque before and after consuming beverage disclosed herein; and

FIG. 4 shows a graphical comparison of exhaled NO. before and after consuming the inventive beverage disclosed herein.

FIG. 5 shows a graphical comparison of brain gauge before and after consuming the inventive beverage disclosed herein.

FIG. 6A and FIG. 6B show a graphical comparison of cognitive scores before and after consuming the inventive beverage disclosed herein; and

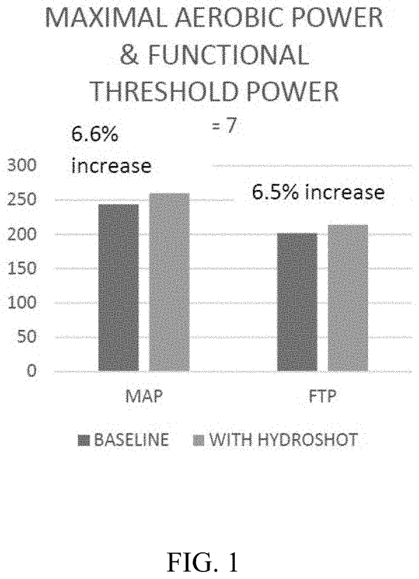

Discussion of FIGS. 1-6 can be found in the EXAMPLES section below.

### DETAILED DESCRIPTION OF THE INVENTION

Illustrative embodiments of the subject matter claimed below will now be disclosed. In the interest of clarity, some features of some actual implementations may not be described in this specification. It will be appreciated that in the development of any such actual embodiments, numerous implementation-specific decisions must be made to achieve the developer's specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

### Definitions

“Degassed potable fluid,” as used herein, means a potable fluid from which volatile compounds and/or solids have been removed by any suitable process, including, but not limited to, reverse osmosis and vacuum distillation.

“Hydrogen,” as used herein, means molecular hydrogen or hydrogen not chemically bonded to any atom other than hydrogen.

“L-citrulline,” as used herein, is an amino acid, naturally occurring in food, such as watermelons and grapefruit, and also produced naturally in the human body as a byproduct of the enzymatic production of nitric oxide from the amino acid arginine, catalyzed by nitric oxide synthase, in particular in the kidneys. Citrulline is a non-essential amino acid, meaning that the body can manufacture it from other nutrients.

“Treated,” as used herein, with respect to a fluid, means that the fluid is degassed to remove volatiles, filtered to remove solids, or both. For example, a treated water can refer to a degassed water, a filtered water, or water that has been both filtered and degassed.

### Beverage Composition

In some embodiments, the present invention relates to a beverage composition comprising the reaction product or product of, a mixture of a hydrogen-infused potable fluid, and a nonessential amino acid. In some embodiments, the beverage composition further comprises one or more of a natural sweetener, a dicarboxylic acid, a tricarboxylic acid, an α-amino acid, an electrolyte, a caffeine composition, and a Vitamin B composition.

Hydrogen-Infused Potable Fluid

The beverages disclosed herein comprise a base potable fluid, which has been infused with hydrogen. In some embodiments, the potable fluid is degassed prior to infusion with hydrogen. In some embodiments, the potable fluid is water, tea, coffee, fruit juice, or water. In some embodiments, the potable fluid is degassed water.

Degassing can be accomplished by any suitable method, such as, but not limited to, reverse osmosis and vacuum distillation. Reverse osmosis is a process by which a solvent passes through a porous membrane in the direction opposite to that for natural osmosis when subjected to a hydrostatic pressure greater than the osmotic pressure. Vacuum distillation is distillation performed under reduced pressure, which allows the purification of compounds not readily distilled at ambient pressures. Regardless of the chosen method for degassing the base potable fluid, degassed potable fluids in some embodiments have a total dissolved solids (“TDS”), as determined by measuring the electrical conductivity of the potable fluid using a common TDS probe, of less than or equal to 150 ppm, 125 ppm, 100 ppm, 75 ppm, or 50 ppm.

In some embodiments, the base potable fluid is water. After degassing, the degassed potable fluid or degassed water is infused with hydrogen. Hydrogen water is made by bubbling pure hydrogen gas into water, by using electrolysis, by using a proton exchange membrane, by reaction with elemental magnesium, or by co-feed of hydrogen and a potable fluid, such as water, through a high-pressure nozzle, wherein the water is atomized into small droplets in the presence of highly concentrated hydrogen to increase hydrogen induction into the potable fluid. In some embodiments, the potable fluid, such as degassed water, is infused with hydrogen in an amount in the range of from 0.5 ppm, 1.0 ppm, 1.5 ppm, 1.6 ppm, 1.7 ppm, 1.8 ppm, 1.9 ppm, 2.0 ppm, 2.1 ppm, 2.2 ppm, 2.3 ppm, 2.4 ppm, or 2.5 ppm to 3.0 ppm, 4.0 ppm, 5.0 ppm, or 6.0 ppm by weight, wherein any listed lower endpoint of the range can be used in conjunction with any listed upper endpoint of the range to form a unique range.

Nonessential Amino Acid

Nonessential amino acids are amino acids produced by the human body even if they are not ingested from food. Nonessential amino acids include and not limited to: alanine, arginine, asparagine, aspartic acid, cysteine, glutamic acid, glutamine, glycine, proline, serine, and tyrosine including all of their enantiomers such as L-tyrosine. Nonessential amino acids support tissue growth and repair, immune function, red blood cell formation, and hormone synthesis. However, unlike essential amino acids, a healthy body can create these proteins if given enough protein sources with essential amino acids.

In some embodiments, the beverage composition comprises molecular hydrogen and a nonessential amino acid, wherein the nonessential amino acid(s) are L-citrulline and/or L-arginine. In some embodiments, the nonessential amino acid(s) added to the mixture forming the beverage composition is in the range of from 0.15 wt %, 0.25 wt %, 0.50 wt %, or 1.0 wt %, to 2.5 wt %, 2.75 wt %, or 3.0 wt %, based on the total weight of the beverage composition. In one embodiment, the nonessential amino acid added to the mixture that makes up the beverage composition is in the range of from 0.15 wt %, to 3.0 wt %, based on the total weight of the mixture.

Natural Sweetener

Natural sweeteners include, but are not limited to, stevia, erythritol, monk fruit, acesulfame potassium, allulose, xylitol, and yacon. In some embodiments, the mixture comprising molecular hydrogen and a nonessential amino acid further comprises a natural sweetener selected from one or more of stevia, erythritol, monk fruit, acesulfame potassium, allulose, xylitol, and yacon. In some embodiments, the natural sweetener is added to the mixture in an amount less than or equal to 4 wt %, 3 wt %, or 2 wt %, based on the total weight of the mixture.

Stevia is extracted from the leaves of a plant called Stevia rebaudiana. Several sweet compounds are found in stevia leaves, primarily stevioside and rebaudioside, both of which are hundreds of times sweeter than sugar, gram for gram. Stevia is very sweet but has virtually no calories. Stevia is believed to be beneficial in lowering high blood pressure and in lowering blood sugar levels in people with diabetes.

Erythritol is a sugar alcohol found naturally in certain fruits and a processed version is available in powdered form. It contains about 6% of the calories in an equal amount of sugar with 70% of the sweetness. Ingestion of erythritol does not spike blood sugar or insulin levels or affect levels of blood fats like cholesterol or triglycerides.

Monk fruit is a type of fruit native to Southeast Asia used to make a natural sweetener called monk fruit extract, which is free of calories and carbohydrates. Some research suggests that it may also help support better blood sugar management. Monk fruit also contains antioxidant compounds known as mogrosides, which studies have shown can reduce markers of inflammation.

Acesulfame potassium is about 200 times sweeter than common sugar.

Allulose is a low-calorie sweetener that has 70% of the sweetness of sugar. People with diabetes and obesity can benefit from this sugar substitute because it is low in calories and has little effect on blood sugar.

Xylitol is a sugar alcohol with a sweetness similar to that of sugar with about two-thirds the calories of sugar. Xylitol is reputed to have some benefits for dental health, to improve bone density, and to help support your gut.

Yacon syrup is harvested from the yacon plant, which grows natively in the Andes in South America. Yacon is very high in fructooligosaccharides, which function as soluble fibers that feed the good bacteria in the intestine.

It is understood that selection of specific or combination of various natural sweeteners can be combined to focus or design a beverage composition of the invention for different uses, effects or treatments.

Dicarboxylic Acid

In some embodiments, the dicarboxylic acid is one or more of adipic acid, fumaric acid, and malic acid. In some embodiments, the dicarboxylic acid is added to the mixture in an amount less than or equal to 2 wt %, based on the total weight of the mixture. In some embodiments, the dicarboxylic acid is malic acid and is added to the mixture comprising molecular hydrogen and a potable fluid, in an amount less than or equal to 2 wt %, based on the total weight of the mixture.

Malic acid is a natural supplement found in fruits like apples that promotes the creation of energy for human cells. Malic acid is an organic compound with the molecular formula C₄H₆O₅. It is a dicarboxylic acid that is made by all living organisms, contributes to the sour taste of fruits, and is used as a food additive. Malic acid has two stereoisomeric forms, though only the L-isomer exists naturally.

Tricarboxylic Acid

In some embodiments, the tricarboxylic acid is one or more of citric acid, isocitric acid, aconitic acid, and propane-1,2,3-tricarboxylic acid. In some embodiments, the tricarboxylic acid is added to the mixture comprising molecular hydrogen and a nonessential amino acid, in an amount less than or equal to 2 wt %, based on the total weight of the mixture. In some embodiments, the tricarboxylic acid is citric acid and is added to the mixture in an amount less than or equal to 2 wt %, based on the total weight of the mixture.

Citric acid is a sharp-tasting crystalline acid present in the juice of lemons and other sour fruits. It is made commercially by the fermentation of sugar and used as a flavoring and setting agent. Citric acid is an organic compound with the chemical formula HOC(CH₂CO₂H)₂. In biochemistry, it is an intermediate in the citric acid cycle, which occurs in the metabolism of all aerobic organisms.

α-Amino Acid

“α-amino acid,” as used herein, means amino acids which have the amine group attached to the carbon atom next to the carboxyl group, or the alpha position. These are also known as 2-, alpha-, or α-amino acids and have a generic formula:

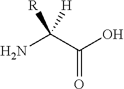

wherein R is an organic substituent (“side chain”).

These are generally L-stereoisomers (“left-handed” isomers), while a few D-amino acids (“right-handed”) occur in bacterial envelopes and in some antibiotics. In some embodiments, the α-amino acid has a hydrophilic nonacidic side chain on the α-carbon, such as, but not limited to serine, threonine, asparagine, and glutamine.

In some embodiments, the α-amino acid is one or more of serine, threonine, asparagine, and glutamine. In some embodiments, the α-amino acid is added to the mixture comprising molecular hydrogen and a nonessential amino acid, in an amount less than or equal to 2 wt %, based on the total weight of the mixture. In some embodiments, the α-amino acid is glutamine and is added to the mixture in an amount less than or equal to 2 wt %, based on the total weight of the mixture.

Electrolyte

In some embodiments, the electrolyte is monopotassium phosphate. In some embodiments, the electrolyte is added to the mixture making up the beverage composition of the invention in an amount less than or equal to 2 wt %, based on the total weight of the mixture.

Monopotassium phosphate is the inorganic compound often used as a fertilizer, food additive, and buffering agent. The salt often cocrystallizes with the dipotassium salt as well as with phosphoric acid.

Caffeine Composition

In some embodiments, the caffeine composition is natural green tea caffeine. In some embodiments, the caffeine composition is added to the mixture making up the beverage composition of the invention in an amount less than or equal to 2 wt %, based on the total weight of the mixture.

Natural green tea caffeine also contains the amino acid L-theanine, which can work synergistically with caffeine to improve brain function 95 to 200 mg. According to a Journal of Food Science test of caffeine levels across commercial brands of green tea, the caffeine content in each 8 oz. cup varied by brand from 11 mg to 47 mg, and other sources record green teas that can contain upwards of 60 mg per brewed cup. Green tea may have a number of health benefits including, but not limited to, assisting in weight management, reducing skin inflammation, treating type 2 diabetes, and improving cardiovascular health. Green tea has one of the highest concentrations of antioxidants of any tea. It is naturally low in calories and contains less caffeine than black tea and coffee. In one embodiment, the beverage composition of the invention comprises a potable fluid, hydrogen, a nonessential amino acid, and green tea.

Vitamin B Composition

In some embodiments, the Vitamin-B composition is selected from one of more of riboflavin (B2), niacin (B3), calcium pantothenate (B5), pyridoxine hydrochloride (B6), biotin (B7), folic acid (B9), and cyanocobalamin (B12). In some embodiments, the Vitamin-B composition is added to the mixture making up the beverage composition of the invention in an amount less than or equal to 2 wt %, based on the total weight of the mixture.

Vitamin B refers to several water soluble vitamins often found together in foods, all of which are necessary for normal growth and metabolism, but none of which are synthesized in adequate amounts by humans. The common forms of vitamin B include vitamin B1 (thiamine), B2 (riboflavin), B3 (niacin), B6 (pyridoxine) and B12 (cyanocobalamin).

Riboflavin (B2) works as an antioxidant, fighting damaging particles in the body known as free radicals. Antioxidants, such as riboflavin, can fight free radicals and may reduce or help prevent some of the damage they cause. Riboflavin is also needed to help the body change vitamin B6 and folate into forms it can use. It is also important for growth and red blood cell production.

Niacin (B3) is used to increase high-density lipoprotein (HDL) “good” cholesterol. Niacin and a related nutrient called niacinamide are used to treat or prevent niacin deficiency.

Calcium pantothenate (B5) is necessary for making blood cells, and it helps you convert the food you eat into energy.

Pyridoxine hydrochloride (B6) is essential to red blood cell, nervous system, and immune systems functions and helps maintain normal blood glucose levels.

Biotin (B7) is involved in a wide range of metabolic processes, both in humans and in other organisms, primarily related to the utilization of fats, carbohydrates, and amino acids.

Folic acid (B9) helps the body make healthy red blood cells and is found in certain foods.

Cyanocobalamin (B12) is important to maintain the health of your metabolism, blood cells, and nerves.

It is understood that any one of the above Vitamin-B can be used alone or in combination with the beverage composition comprising a potable fluid, molecular hydrogen and a nonessential amino acid.

Other Beverage Embodiments

In some embodiments, the composition comprises the product or reaction product of a mixture of hydrogen-infused, degassed water, L-citrulline, erythritol, stevia, malic acid, citric acid, L-glutamine, monopotassium phosphate, riboflavin (B2), natural green tea caffeine, niacin (B3), calcium pantothenate (B5), pyridoxine hydrochloride (B6), biotin (B7), folic acid (B9), and cyanocobalamin (B12).

In some embodiments, the composition comprises the product or reaction product of a mixture of:
a treated water infused with hydrogen in an amount in the range of from 0.5 ppm to 6 ppm by weight;
L-citrulline in an amount in the range of from 0.15 wt % to 3.0 wt %;
erythritol, stevia; malic acid, citric acid, L-glutamine, monopotassium phosphate, and natural green tea caffeine, each in an amount less than or equal to 2 wt %;
riboflavin (B2), in an amount less than or equal to 1300 ppm by weight; niacin (B3), in an amount less than or equal to 16,000 ppm by weight;
calcium pantothenate (B5), in an amount less than or equal to 5000 ppm by weight;
pyridoxine hydrochloride (B6), in an amount less than or equal to 1300 ppm by weight;
biotin (B7), in an amount less than or equal to 30 ppm by weight;
folic acid (B9), in an amount less than or equal to 400 ppm by weight; and
cyanocobalamin (B12), in an amount less than or equal to 2.4 ppm by weight;
wherein all weights are based on the weight of the mixture.

Other Beverage Embodiments

In some embodiments, additional ingredients in the beverage composition include potassium, vitamin C, and cannabidiol.

### Process of Making Beverage Composition

In some embodiments, the process of making the beverage composition comprises infusing a potable fluid with hydrogen to produce a hydrogen-infused potable fluid and mixing the hydrogen-infused potable fluid with a nonessential amino acid, such as, but not limited to, L-citrulline, to produce an enhanced beverage composition of the invention.

In some embodiments, the process further includes degassing the potable fluid to remove volatiles and/or solids prior to infusing the potable fluid with hydrogen.

The process of either of the preceding process embodiments, further comprising mixing the enhanced beverage composition of the invention with one or more of a natural sweetener, a dicarboxylic acid, a tricarboxylic acid, an α-amino acid, an electrolyte, a caffeine composition, and a Vitamin-B composition.

### Beverage Advantages

Nitric oxide is a colorless, tasteless short-acting gas helps blood vessels dilate, which in turn leads to an increased oxygen flow throughout the body. Nitric oxide, also called nitrogen oxide or nitrogen monoxide, is produced by almost every cell of the human body. Two amino acids, L-arginine and L-citrulline, boost nitric oxide production in the body. More specifically, the kidneys turn L-citrulline into L-arginine, which is a precursor to nitric oxide. Also, nitric oxide is produced in the human body from foods that contain nitrates, which are then converted to nitrites. Nitrites then are converted into nitric oxide in the body. Roughly 80 percent of dietary nitrates come from eating vegetables.

Nitric oxide is a gas that improves blood flow in areas of the lungs that are getting air, increasing the amount of oxygen in the blood stream. Nitric oxide plays key roles in maintaining normal vascular function and regulating inflammatory cascades that contribute to acute lung injury and acute respiratory distress syndrome. Nitric oxide also helps to support a healthy heart, boost the immune systems and increase exercise performance.

The main issue with nitric oxide is that it has a very short life and burns off very rapidly in the human body.

When adding at least 0.5 grams of a vegetable and/or fruit which contains nitrates or one of either of the amino acids, L-arginine or L-citrulline, and adding hydrogen with the aforementioned ingredients, the following new benefits can be achieved: increased blood flow that lasts for over 4 hours; limited to no increase in heart rate; limited to no increase in blood pressure; improved immune system; reduction of inflammation; increase sports performance; increased focus; and increased endurance.

Some disorders or physiological conditions can be medicated by promoting the production of nitric oxide in the human body. Increasing the production of nitric oxide can prevent, reverse, or limit the progression of disorders which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism and idiopathic or primary pulmonary hypertension or chronic hypoxia. However, nitric oxide can also induce lethal cellular injury under stressed conditions. Advantageously, molecular hydrogen helps regulate nitric oxide production and attenuates harmful effects. However, the inventive beverage composition is useful in treating the above disorders or physiological conditions. While the inventive beverage is not a cure, it is believed that the beneficial effects of extended nitric oxide release in the human body as a result of administering, drinking, the inventive beverage composition, can improve, reduce or minimize the listed disorders and physiological conditions. In a preferred embodiment it has been found that the beneficial effect of the beverage composition are enhanced when the beverage composition is consumed prior to ingesting solid food. It is best to drink the inventive beverage on an empty stomach.

The following examples illustrate the invention; however, those skilled in the art will recognize numerous variations within the spirit of the invention and scope of the claims. To facilitate a better understanding of the present invention, the following examples of preferred embodiments are given. In no way should the following examples be read to limit, or to define, the scope of the invention.

### Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Physical Performance Testing

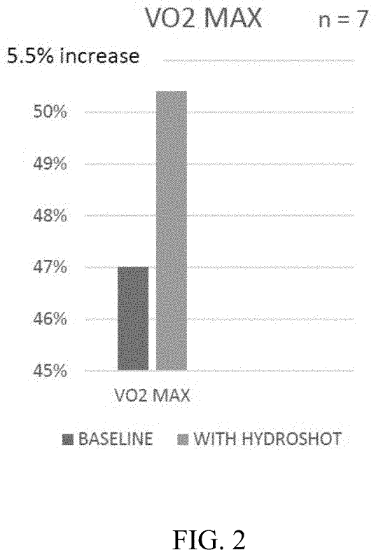

Testing was setup to determine possible improvements in maximal aerobic power (MAP), functional threshold power (FTP), and maximal oxygen uptake (VO2) using cycle ergometer. Data is from drinking Hydro Shot vs baseline without Hydro Shot. Data generated at corpus performance sports facility in Dallas, Tex. Seven (7) subjects were included in the tests. Results are shown in FIG. 1 and FIG. 2.

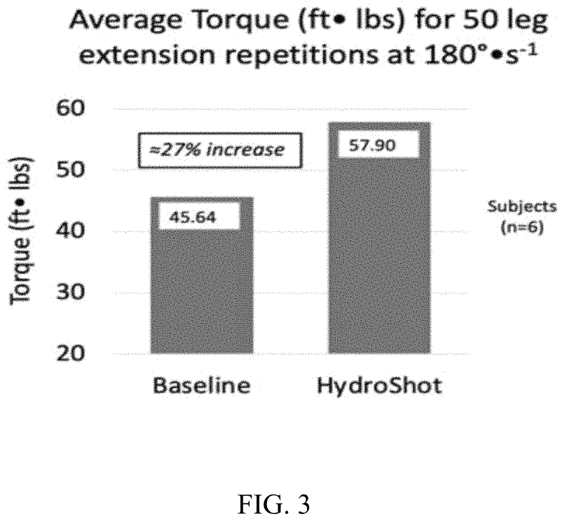

Testing was setup to determine possible improvements in leg extension repetition performance obtained from drinking Hydro Shot vs baseline without Hydro Shot. Data was generated at the Department of Kinesiology at Southern Utah University. Six (6) subjects were included in the tests. Results are shown in FIG. 3.

### Cognitive Testing

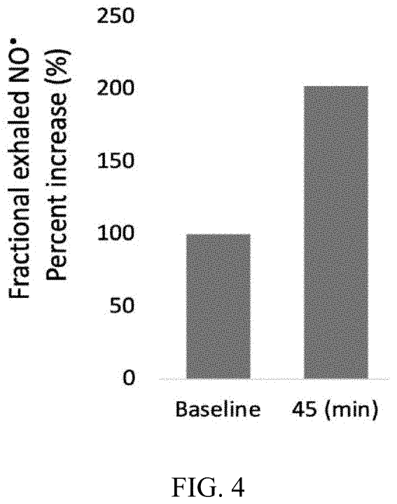

Testing was to determine possible sports performance improvements for speed, accuracy and focus drinking Hydro Shot vs baseline without drinking Hydro Shot. Data was generated using cortical metric's brain gauge tool at Corpus Performance Sports Facility in Dallas, Tex. Ten (10) subjects were included in the tests. Results are shown in FIG. 4.

The foregoing examples and related FIGS. 1-4 and results are published with additional details in: Effects of an H2-infused, Nitric Oxide-Producing Functional Beverage on Exercise and Cognitive Performance, LeBaron, Kharman, and McCullough, The Journal of Science and Medicine, Vol. 3, No. 2 (2021), and A novel functional beverage for COVID-19 and other conditions: Hypothesis and preliminary data, increased blood flow, and wound healing, LeBaron, Kharman, and McCullough, Journal of Translational Science, Vol. 6, doi: 10.15761/JTS.1000380, ISSN: 2059-268X (2020)—the entire contents of both document fully incorporated by reference herein.

### Traumatic Brain Injury Testing

Traumatic brain injuries may be caused by injuries from a number of sports, including soccer, boxing, football, baseball, lacrosse, skateboarding, hockey, and other high impact or extreme sports. These are particularly common in youth, explosive blasts, and other combat injuries.

An estimated 300,000 sports-related traumatic brain injuries (TBIs) of mild to moderate severity, most of which can be classified as concussions (i.e., conditions of temporarily altered mental status as a result of head trauma), occur in the United States each year. the proportion of these concussions that are repeat injuries is unknown. However, there is an increased risk for subsequent TBI among persons who have had at least one previous TBI.

Five-year outcomes of persons with TBI: 22% died, 30% became worse, 22% stayed same, and 26% improved. Data are us population estimates based on the TBIMS national database. Data refer to people 16 years of age and older who received inpatient rehabilitation services for a primary diagnosis of TBI.

Traumatic Brain Injury Diagnostic Methods include but not limited to:
MRI
Doppler Ultrasound
CTN Scan
UC Davis FIDWS
Impact
Cortical Metrics Brain Gauge

Beverages of the invention disclosed herein is useful for one or more therapeutic for TBI including:
Increases blood flow
Reduces inflammation
Improves focus
Improves memory
Boosts nitric oxide production for hours

Impact testing results before and after drinking hydrogen infused beverage disclosed herein:

| | Before Hydrogen- | Before Hydrogen-infused |
|---|---|---|
| | infused beverages | beverages disclosed |
| | disclosed herein | herein |
| Test Method | (Hydro Shot) | (Hydro Shot) |
| Designed Memory | 60% | 77% |
| Learning Percent Correct | 63% | 83% |
| Percent of Letters Correct | 93% | 100% |
| Delayed Memory Percent | 58% | 71% |
| Correct | | |
| 3 Letters Counted Correctly | 6% | 11.2% |

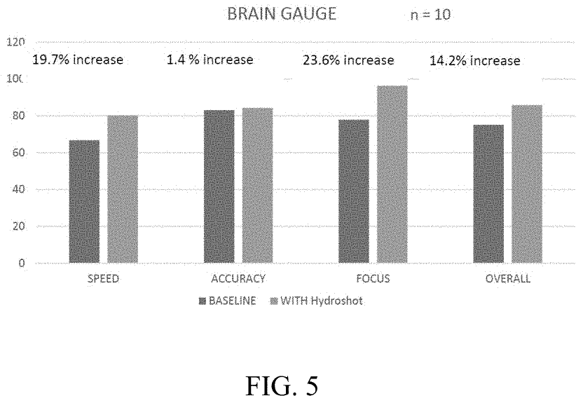

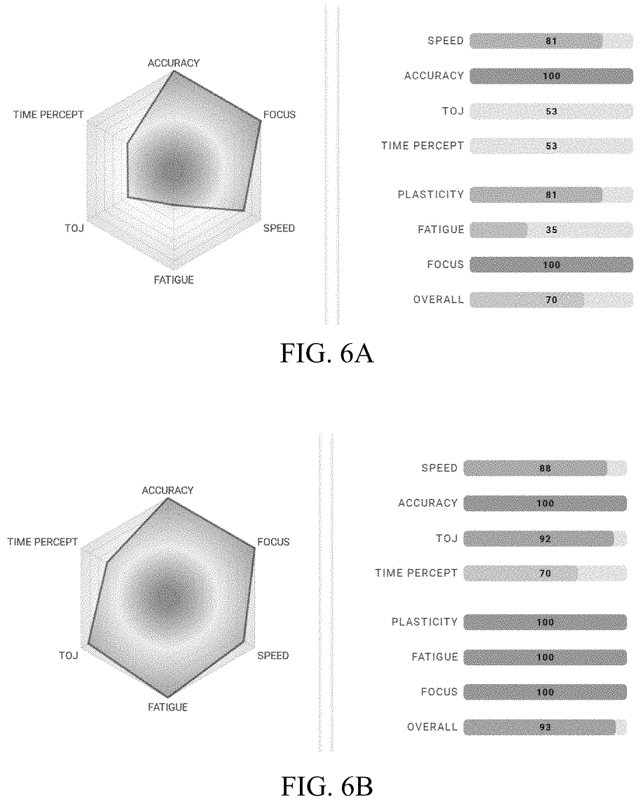

Cortical metrics brain gauge test results before and after drinking Hydro Shot are shown in FIGS. 5, 6A and 6B.

### Nitric Oxide Production

Subjects were tested for increased nitric oxide production following ingestion of product. Baseline (time=0) showed depleted nitric oxide levels, while 0.5 hrs., 1 hr., 3 hrs., 5 hrs., and 8 hrs. after ingestion Hydro Shot showed optimal nitric oxide levels.

Increased nitric oxide production followed ingestion of product. FIG. 7 shows the average percent increase of FeNO. by 12 subjects following product ingestion after 45 min compared to baseline.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the compositions, processes, and/or steps described in the specification. As one of the ordinary skill in the art will readily appreciate from the disclosure of the present invention, compositions, processes, and/or steps, presently existing or later to be developed that perform substantially the same function or achieve substantially the same result as the corresponding embodiments described herein, may be utilized according to the present invention. Accordingly, the appended claims are intended to include within their scope such compositions, processes, and/or steps.

What is claimed is:

1. A composition comprising a mixture of:
a. a potable fluid; and
b. a nonessential amino acid;
wherein the potable fluid and/or the mixture are infused with molecular hydrogen.

2. The composition of claim 1 wherein the potable fluid is a treated potable fluid.

3. The composition of claim 1 wherein the potable fluid is water and the potable fluid and/or the mixture are infused with molecular hydrogen in an amount in the range of from 0.5 ppm to 6 ppm by weight.

4. The composition of claim 1 wherein the nonessential amino acid is L-citrulline and/or L-arginine.

5. The composition of claim 1 wherein the amount of nonessential amino acid is in the range of from 0.15 wt % to 3.0 wt %, based on the total weight of the composition.

6. The composition of claim 1 wherein the mixture further comprises one or more members selected from the list consisting of: a natural sweetener, a dicarboxylic acid, a tricarboxylic acid, an α-amino acid, an electrolyte, a caffeine composition, and a Vitamin-B composition.

7. The composition of claim 6 wherein the natural sweetener is one or more members selected from the list consisting of: stevia, erythritol, monk fruit, acesulfame potassium, allulose, xylitol, and yacon.

8. The composition of claim 6 wherein the natural sweetener is in an amount less than or equal to 4 wt %, based on the total weight of the mixture.

9. The composition of claim 6 wherein the dicarboxylic acid is one or more members selected from the list consisting of: adipic acid, fumaric acid, and malic acid.

10. The composition of claim 6 wherein the dicarboxylic acid is in an amount less than or equal to 2 wt %, based on the total weight of the mixture.

11. The composition of claim 6 wherein the tricarboxylic acid is citric acid.

12. The composition of claim 6 wherein the tricarboxylic acid is in an amount less than or equal to 2 wt %, based on the total weight of the mixture.

13. The composition of claim 6 wherein the α-amino acid is L-glutamine.

14. The composition of claim 6 wherein the α-amino acid is in an amount less than or equal to 2 wt %, based on the total weight of the mixture.

15. The composition of claim 6 wherein the electrolyte is monopotassium phosphate.

16. The composition of claim 6 wherein the electrolyte is in an amount less than or equal to 2 wt %, based on the total weight of the mixture.

17. The composition of claim 6 wherein the caffeine composition is natural green tea.

18. The composition of claim 6 wherein the caffeine composition is in an amount less than or equal to 2 wt %, based on the total weight of the mixture.

19. The composition of claim 6 wherein the Vitamin-B composition is selected from one of more of the list consisting of: riboflavin (B2), niacin (B3), calcium pantothenate (B5), pyridoxine hydrochloride (B6), biotin (B7), folic acid (B9), and cyanocobalamin (B12).

20. The composition of claim 6 wherein the Vitamin-B composition is in an amount less than or equal to 2 wt %, based on the total weight of the mixture.

21. The composition of claim 1 wherein the nonessential amino acid is L-citrulline and/or L-arginine and the mixture further comprises one or more of the group consisting of: erythritol, stevia, malic acid, citric acid, L-glutamine, monopotassium phosphate, riboflavin (B2), natural green tea caffeine, niacin (B3), calcium pantothenate (B5), pyridoxine hydrochloride (B6), biotin (B7), folic acid (B9), and cyanocobalamin (B12).

22. The composition of claim 1 wherein the mixture further comprises cannabidiol.

23. A beverage composition comprising the mixture of:
a. a potable fluid comprising molecular hydrogen in an amount in the range of from 0.5 ppm to 6 ppm by weight;
b. L-citrulline in an amount in the range of from 0.15 wt % to 3.0 wt %;
c. erythritol, stevia; malic acid, citric acid, L-glutamine, monopotassium phosphate, and natural green tea caffeine, each in an amount less than or equal to 2 wt %;
d. riboflavin (B2), in an amount less than or equal to 1300 ppm by weight;
e. niacin (B3), in an amount less than or equal to 16,000 ppm by weight;
f. calcium pantothenate (B5), in an amount less than or equal to 5000 ppm by weight;
g. pyridoxine hydrochloride (B6), in an amount less than or equal to 1300 ppm by weight;
h. biotin (B7), in an amount less than or equal to 30 ppm by weight;
i. folic acid (B9), in an amount less than or equal to 400 ppm by weight; and
j. cyanocobalamin (B12), in an amount less than or equal to 2.4 ppm by weight;
wherein:
all weights are based on the weight of the mixture; and
the potable fluid and/or the mixture are infused with molecular hydrogen.

24. The beverage composition of claim 23 wherein in the potable fluid is water or treated water.

25. The beverage composition of claim 23 wherein potable fluid and/or the mixture are infused with molecular hydrogen in an amount of greater than 1.8 ppm to 6 ppm by weight.

26. A process for making a beverage composition comprising the steps of:
a. mixing a potable fluid with a nonessential amino acid to produce an enhanced beverage; and
b. infusing hydrogen into:
i. the potable fluid prior to mixing, and/or
ii. the enhanced fluid after mixing.

27. The process of claim 26 further comprising the step of treating the potable fluid before mixing.

28. The process of claim 26 where in the potable fluid is water, and the nonessential amino acid is L-citrulline and/or L-arginine.

29. The process of claim 26 wherein level of hydrogen is added in an amount such that beverage composition contains hydrogen in an amount in the range of from 2 ppm and 6 ppm by weight,

30. The process of claim 26 wherein the infusing comprises the use of a high-pressure nozzle.

31. A method for treatment of a patient having a medical condition, the method comprising: administering a beverage composition comprising a mixture of a hydrogen-infused potable fluid and a nonessential amino acid, where the medical condition is selected from one of the group consisting of: acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma, status asthmaticus or hypoxia, chronic pulmonary hypertension, bronchopulmonary dysplasia, chronic pulmonary thromboembolism, idiopathic or primary pulmonary hypertension and chronic hypoxia.

32. The method of claim 31 wherein the potable fluid is water and the nonessential amino acid is L-citrulline.

33. The method of claim 31 wherein the amount of hydrogen contained in the beverage composition is in the range of greater than 1.7 ppm to about 6 ppm by weight.

34. The method of claim 31 wherein the amount of hydrogen contained in the beverage composition is in the range of form greater than 2 ppm to about 6 ppm by weight.

35. The method of claim 31 wherein the amount of hydrogen contained in the beverage composition is in the range of from 2.5 ppm to 5 ppm by weight.

36. The method of claim 31 wherein the administering takes place prior to the ingestion of any solid food.
